(19) United States
(12) Patent Application Publication
(10) Pub. No.: US 2015/0337372 A1
(43) Pub. Date: Nov. 26, 2015

(54) METHODS AND APPARATUS FOR SELECTIVELY PROCESSING EGGS ACCORDING TO GENDER AND OTHER CHARACTERISTICS

(71) Applicant: Zoetis Services LLC, Florham Park, NJ (US)

(72) Inventors: Theodore Medard Winger, Morrisville, NC (US); Michael Peter Robinson, Chapel Hill, NC (US); Stephen Penn Wolfe, Chapel Hill, NC (US); Amanda Elizabeth Basciano, Apex, NC (US)

(21) Appl. No.: 14/719,377

(22) Filed: May 22, 2015

Related U.S. Application Data
(60) Provisional application No. 62/139,903, filed on Mar. 30, 2015.
(60) Provisional application No. 62/001,791, filed on May 22, 2014.

(51) Int. Cl. C12Q 1/68; G01N 33/74; G01N 1/02

(52) U.S. Cl. CPC C12Q 1/6879; G01N 1/02; C12Q 1/6806; G01N 33/743; C12Q 2600/124

(57) ABSTRACT

Methods and apparatuses for processing eggs based upon a characteristic such as gender are provided. Sample material is extracted from each of a plurality of live eggs. The extracted sample material is deposited on a liquid-immobilizing assay template and then assayed to identify eggs having the characteristic. The eggs identified as having the characteristic are processed accordingly.

### CROSS REFERENCE TO RELATED APPLICATIONS

This application claims the benefit of U.S. Provisional Patent Application Nos. 62/001,791, filed May 22, 2014, and 62/139,903, filed Mar. 30, 2015, both of which are expressly incorporated herein by reference in their entirety.

### TECHNICAL FIELD

The present invention relates generally to eggs and, more particularly, to methods and apparatuses for pre-hatch processing of eggs having viable embryos.

### BACKGROUND

Discrimination between poultry eggs (hereinafter “eggs”) on the basis of some observable quality is a well-known and long-used practice in the poultry industry. “Candling” is a common name for one such technique, a term which has its roots in the original practice of inspecting an egg using the light from a candle. Although egg shells appear opaque under most lighting conditions, eggs are actually somewhat translucent. Accordingly, when placed in front of a light, the contents of an egg can be observed.

In poultry hatcheries, one purpose of candling eggs is to identify and then segregate live eggs (i.e., eggs which are to be hatched to live poultry) from non-live eggs (e.g., clear eggs, dead eggs, rotted eggs, empty eggs, etc.). Once identified, live avian eggs may be treated with medications, nutrients, hormones and/or other beneficial substances while the embryos are still in the egg (i.e., in ovo). In ovo injections of various substances into avian eggs have been employed to decrease post-hatch morbidity and mortality rates, increase the potential growth rates or eventual size of the resulting bird, and even to influence the gender determination of the embryo. Injection of vaccines into live eggs have been effectively employed to immunize birds in ovo.

In ovo injection of a virus may be utilized to propagate the particular virus for use in preparation of vaccines. Examples of substances that have been used for, or proposed for, in ovo injection include vaccines, antibiotics and vitamins.

In commercial hatcheries, eggs typically are held in setting flats during incubation. At a selected time, typically on the eighteenth day of incubation, the eggs are removed from an incubator. Unfit or non-viable eggs (namely, dead eggs, rotted eggs, empties, and clear eggs) are identified and removed, live eggs are treated (e.g., inoculated) and then transferred to hatching baskets.

In hatchery management, it may be desirable to separate birds based upon various characteristics, such as gender, diseases, genetic traits, etc. For example, it may be desirable to inoculate male birds with a particular vaccine and inoculate female birds with a different vaccine. Sex separation of birds at hatch may be important for other reasons as well. For example, turkeys are conventionally segregated by sex because of the difference in growth rate and nutritional requirements of male and female turkeys. In the layer or table egg industry, it is desirable to keep only females. In the broiler industry, it is desirable to segregate birds based on sex to gain feed efficiencies, improve processing uniformity, and reduce production costs.

Unfortunately, conventional methods of sexing birds may be expensive, labor intensive, time consuming, and typically require trained persons with specialized skills. Conventional methods of sexing birds include feather sexing, vent sexing, and DNA or blood sexing. DNA or blood sexing is performed by analyzing a small sample of blood collected from a bird.

It would be desirable to identify the sex of birds, as well as other characteristics of birds, prior to hatching. Pre-hatch sex identification could reduce costs significantly for various members of the poultry industry.

### SUMMARY OF THE INVENTION

In view of the above discussion, aspects of the present disclosure provide methods of processing eggs having an identified characteristic (e.g., gender) wherein material (e.g., allantoic fluid, amnion, yolk, shell, albumen, tissue, membrane and/or blood, etc.) is extracted from each of a plurality of live eggs, the extracted material is assayed to identify eggs having a characteristic, and then eggs identified as having the characteristic are processed accordingly. For example, a method of processing eggs based upon gender, according to aspects of the present disclosure, includes identifying live eggs among a plurality of eggs, extracting material from the eggs identified as live eggs, assaying each live egg to identify a gender of each live egg, and selectively injecting a vaccine into the live eggs according to gender.

According to aspects of the present disclosure, an automated gender sorting system is provided and includes three independent modules linked via a network. The first module is an material sampling module. Flats of eggs are removed from a setting incubator at a predetermined time during an incubation cycle and fed onto a conveyor belt. A candling system automatically identifies live eggs and the eggs (either only live eggs or all eggs) are transferred to a sampling station. A needle is then inserted into each egg and material is withdrawn. The material sample from each egg is deposited onto an assay template having a spatially-immobilizing property toward liquids such that the liquid is immobilized within a component of the assay template through absorption (hereinafter, referred to as “liquid-immobilizing”). According to some aspects, a liquid-immobilizing assay template has an absorbent or hydrophilic substrate patterned with hydrophobic barriers so as to form a plurality or array of assay locations. The deposited material samples may be arranged in the same array as the array of the egg flat, according to aspects of the present disclosure. Each sampling needle is sanitized before being used to sample material from another egg. The flats are then returned to a setting incubator or a holding station. The assay template containing the sampled material from the eggs are queued for processing.

The second module is an automated assaying module. The assay templates containing sampled material are transported into the assaying module. Within the assaying module, each assay template is moved beneath a dispensing head which dispenses a predetermined amount of reagent onto each respective assay location (e.g., a well, an absorbent pad, or any other means of locating a liquid sample in a 2-or 3-dimensional arrangement). Each assay template then progresses through an environmentally-controlled chamber for a predetermined period of time. The characteristic, such as, for example, gender, of a respective egg whose sample material is at the assay location is then determined. This information is stored via a data processor on the network.

The third module is an egg treatment and sorting module. According to aspects of the present disclosure, a data processor on the network identifies which eggs are male and which eggs are female based on information previously stored. The male eggs are then vaccinated with a male-specific vaccination and the female eggs are vaccinated with a female-specific vaccination. According to aspects of the present disclosure, separate vaccination devices may be utilized for male and female eggs. Once vaccinated, the eggs are sorted by gender and transferred to gender-specific hatching baskets. The hatching baskets are then transferred to hatching incubators. According to aspects of the present disclosure, eggs of one gender can be discarded and not vaccinated or transferred into hatching baskets.

Aspects of the present disclosure can facilitate increased production efficiencies by contributing to savings in incubation space (e.g., not hatching chicks identified as males pre-hatch), by contributing to savings in vaccinations, by allowing reduction in manual labor, and by increasing hatchery processing speeds. Because the gender of each egg is known prior to vaccination, savings in vaccination costs can be realized particularly when it is desirable to vaccinate only a specific gender.

### BRIEF DESCRIPTION OF THE DRAWINGS

FIG. 1 is a schematic perspective view of an egg processing system, according to aspects of the present disclosure;

FIG. 2 is a schematic perspective view of an egg sampling device capable of extracting a sample from a plurality of eggs and depositing the samples on an assay template, according to aspects of the present disclosure;

FIGS. 3-4 illustrate assay templates for use in an egg processing system, according to aspects of the present disclosure;

FIG. 5 illustrates a sealed assay template, according to aspects of the present disclosure;

FIGS. 6 and 7 illustrate assay templates for use in an egg processing system, according to aspects of the present disclosure;

FIG. 8 illustrates a plurality of assay templates disposed on a carrier substrate, according to aspects of the present disclosure;

FIGS. 9-11 are schematic perspective views of a press device capable of introducing moisture into an assay template, according to aspects of the present disclosure;

FIG. 12 illustrates a processing sequence for an egg processing system using an absorbent assay template;

FIGS. 13-21 illustrate another processing sequence for an egg processing system using an absorbent assay template; and

FIG. 22 illustrates a schematic cross-sectional view of a capillary film used as an assay template.

### DETAILED DESCRIPTION OF THE INVENTION

Various aspects of the present disclosure now will be described more fully hereinafter with reference to the accompanying drawings, in which some, but not all aspects of the disclosure are shown. Indeed, this disclosure may be embodied in many different forms and should not be construed as limited to the aspects set forth herein; rather, these aspects are provided so that this disclosure will satisfy applicable legal requirements. Like numbers refer to like elements throughout.

Methods and apparatuses according to aspects of the present disclosure may be utilized for identifying one or more characteristics of an egg at any time during the embryonic development period (also referred to as the incubation period) thereof. Aspects of the present disclosure are not limited to a particular day during the embryonic development period.

Methods of processing live eggs based upon identified characteristics, according to aspects of the present disclosure, are illustrated. Initially, live eggs may be identified among a plurality of eggs undergoing incubation. For example, the eggs may be candled to identify which eggs are live eggs. Material may be extracted from each live egg and the extracted material assayed to identify one or more characteristics (e.g., gender, pathogen content, genetic markers related to bird health or performance, nutritional, endocrine or immune indicators or factors, etc.) of the respective egg. The live eggs may then be selectively processed based upon the identified one or more characteristics.

Identifying live eggs among a plurality of eggs may involve various techniques including, but not limited to, conventional candling (opacity), spectral candling, heartbeat candling, or thermal candling. Aspects of the present disclosure may utilize any method of determining whether an egg contains a live embryo, and are not limited to only the methods described herein.

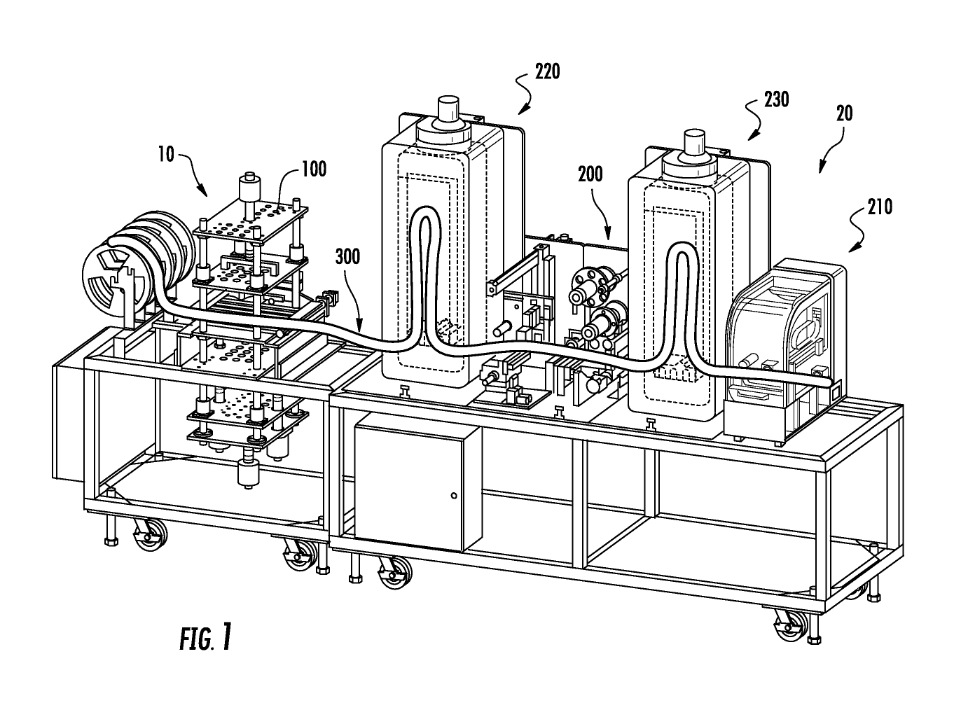

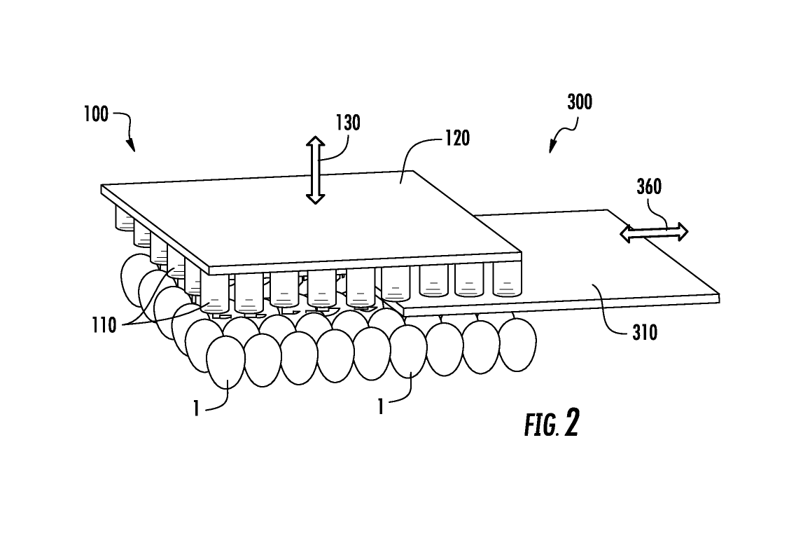

Referring to FIGS. 1 and 2, operations for extracting material from live eggs, according to aspects of the present disclosure, will now be described with respect to sampling module 10 having a sampling station 100. A plurality of live eggs may be positioned in a generally vertical orientation. A probe (e.g., a needle, etc.) of a sampling device 110 may be inserted into each egg through the shell of the egg. The precise location and angle of insertion of a sampling device 110 within an egg may be a matter of choice and could be in any area of an egg. Orientation of a sampling device 110 may depend on the orientation of the egg, the equipment available to carry out the material extraction, as well as the purpose of the material extraction. The extracted material may be of various components of an egg. Various materials (e.g., amnion, yolk, shell, albumen, tissue, membrane and/or blood, etc.) may be extracted from an egg and assayed to identify one or more characteristics, as described below. A sample may be withdrawn from each egg and then the eggs moved to another location for subsequent processing.

With continuing reference to FIG. 1, operations for assaying material extracted from each live egg to determine one or more characteristics of the egg, such as gender, according to aspects of the present disclosure, will now be described. Material, such as whole blood, may be extracted from each egg and dispensed into respective sample receptacles or assay wells in an assay template. A biosensor or reagent, which is configured to chemically react with extracted material and produce detectable signals (e.g., electromagnetic signals, luminescence signals, fluorescence signals, conductivity signals, colormetric signals, pH signals, etc.), may be dispensed into the respective sample receptacles using a dispense station 200 of an assaying module 20.

The presence of a characteristic of an egg is then detected using a detector station 210. Operations of the detector station 210 may be intended to include detection of electromagnetic signals produced within the sample receptacles which provide an indication of the presence of a characteristic of an egg. According to other aspects of the present disclosure, operations of the detector stations 210 may be intended to include detection of pathogens in extracted egg material. One or more additional analyses may be performed on the extracted egg material in the sample receptacles. For example, genetic analysis may be performed on the material.

Operations for selectively processing live eggs based upon identified characteristics may be implemented, according to aspects of the present disclosure. One or more substances may be injected in ovo based upon identified characteristics of each egg. For example, a vaccine may be injected into eggs according to gender of the eggs. Moreover, a first vaccine may be injected into eggs identified as male, and a second vaccine may be injected into eggs identified as female. In addition, the live eggs may be sorted according to identified characteristics. For example, if the identified characteristic is gender, male eggs may be segregated from female eggs.

Sorting may occur before, after, or in lieu of in ovo injection or other treatment or processing. In some instances, eggs may be sorted by gender first and then injected with one or more substances based on gender (e.g., males can be inoculated with a substance and females can be inoculated with a different substance and/or at different times).

The sampling module 10 and assaying module 20 illustrated in FIGS. 1 and 2 may be implemented in an egg processing system for processing eggs, according to aspects of the present disclosure. The system may include a classifier configured to identify live eggs from among a plurality of eggs 1 in an incoming egg flat. The classifier may be operatively connected to a controller which controls the classifier and stores information about each egg 1 (e.g., whether an egg is live (viable) or non-live (non-viable)). As described above, the classifier may include a conventional candling system, a spectral candling system, a candling system that utilizes the combination of light and thermal candling, or any other apparatus/technique for identifying live eggs and/or non-live eggs. An operator interface (e.g., a display) may be provided to allow an operator to interact with the controller.

A material extraction station (also referred to as the sampling station) 100, egg treatment station, and egg sorting station may be provided downstream of the classifier and may each be operatively connected to the controller. The assaying module 20 may also be operatively connected to the controller. The material extraction station 100 may be configured to extract material, such as blood, from selected eggs. Material extracted from each egg may be analyzed via the assaying module 20 to identify one or more characteristics of each egg or for diagnostic or other purposes. For example, the gender of each egg may be identified by analyzing material extracted from an egg. Alternatively, the presence of pathogens may be detected, and/or various genetic analyses may be performed on the extracted material.

The treatment station may be configured to treat selected eggs for example, by inoculation with a treatment substance (e.g., vaccines, nutrients, etc.). The controller may generate a selective treatment signal for an egg (or a group of eggs) based upon characteristics of an egg (or a group of eggs) identified via the assaying module 20. For example, eggs identified as female may be injected with a particular vaccine via the treatment station upon receiving a treatment signal from the controller.

The sorting station may be configured to sort eggs based upon identified characteristics. The controller generates a selective sorting signal for an egg (or a group of eggs) based upon characteristics of an egg (or a group of eggs) identified via the assaying module 20. For example, eggs identified as male may be placed in a first hatching bin, and eggs identified as female may be placed in a second hatching bin.

The assaying module 20 may be configured to perform various tests on material extracted from eggs in order to identify one or more characteristics (e.g., gender) of each egg. Various tests may be performed via the assaying module 20. The present disclosure is not limited only to identifying the gender of eggs.

The controller may include a processor or other suitable programmable or non-programmable circuitry including suitable software. The controller may also include such other devices as appropriate to control the material extraction station 100, egg treatment station, egg sorting station, and assaying module 20. Suitable devices, circuitry and software for implementing a controller will be readily apparent to those skilled in the art upon reading the foregoing and following descriptions.

The operator interface may be any suitable user interface device and preferably includes a touch screen and/or keyboard. The operator interface may allow a user to retrieve various information from the controller, to set various parameters and/or to program/reprogram the controller. The operator interface may include other peripheral devices, for example, a printer and a connection to a computer network.

According to aspects of the present disclosure, one or more of the stations or modules described with respect to FIGS. 1 and 2 may be controlled by individual programmable logic controllers (PLCs). Data may be transferred back and forth from a PLC to a central computer database controller for storage. For example, a central database may be provided to store information such as gender (as well as other identified characteristics) of eggs being processed. The central computer database controller may be configured to respond to individual PLCs when they request data or send data. The central computer database need not directly control the various stations under the control of respective PLCs.

With reference to FIG. 2, a material extraction station or sampling station 100 for extracting material from a plurality of eggs, according to aspects of the present disclosure, is illustrated. The material extraction station 100 may receive eggs via an egg flat conveyor system. In some instances, the material extraction station 100 may also include a classifier configured to identify live eggs from among a plurality of eggs, an assay template handling system, sampling devices 110, and a sanitizer system (not shown), for sanitizing sampling portions of the sampling devices 110. Further, in some instances, the material extraction station 100 may be capable of providing a stream or puff of air to dry a needle of the sampling device after the needle is sanitized with sanitizing fluid.

The egg flat conveyor system may be configured to transport incoming flats of eggs 1 through the classifier. Eggs determined as live may remain in the flat, while eggs determined to be non-live may be removed from the flats such that only live eggs (i.e., those determined to have a live embryo) are transported to the sampling station 100. The egg flat conveyor system, according to an aspects of the present disclosure, may then be configured to transport flats of eggs that have had material extracted therefrom to an incubator for incubation, and/or to subsequent treatment and/or sorting stations.

The sampling station 100 may include an array of sampling devices 110 that are configured to extract material from a respective egg positioned therebeneath. Each sampling device 11 may be configured for generally vertical movement relative to the egg flat. In some instances, a sampling head 120 having the sampling devices 110 may be capable of vertical movement in either direction along line 130.

The eggs from which material has been extracted may be transferred to an incubator for incubation according to conventional procedures while awaiting results from the assaying module 20. When assaying results are completed, and characteristics of each egg identified (e.g., gender) the eggs may be moved from the incubator to one or more treatments stations and/or to a sorting station. According to aspects of the present disclosure, the assaying module 20 may be connected to the material extraction station 100 and may be configured to assay material extracted from eggs quickly. As such, flats of eggs from which material has been extracted may be held in one or more accumulation modules instead of being returned to incubators prior to being transported to a treatment/sorting station(s).

Each sampling device 110 may be configured to extract material from an egg and deposit the extracted material within a respective sample receptacle or assay well in an assay template 300, which may be continuous or discontinuous. That is, when an egg flat containing eggs 1 is positioned beneath the sampling station 100, each sampling device 110 may be configured to extract material from a respective egg 1 and then deposit the extracted material into a respective sample receptacle of the assay template 300.

Each sampling device 110 may include an elongated housing and an elongated needle disposed therewithin and movable between a refracted position and an extended position. In some instances, the needle, when in the extended position, may be configured to punch through the shell of an egg and extract material (e.g., blood) from the egg. The needle may then be configured to deliver extracted egg material into a respective sample receptacle of the assay template 300, as will be described below. For dispensing extracted material into a sample receptacle, a sample tray may be moved upwardly to the needle or laterally therebeneath, as shown in FIG. 2 (along directional line 360). In some instances, the dispensed sample material may include additional substances such as, for example, NaOH, buffers (to control pH and/or ionic strength), surfactants, adjuvants, viscosity modifiers (flow control), non-aqueous based media such as organic solvents, ionic liquids (e.g., imidazolium family and the like), deep eutectic solvents (DES), silica gel, micro or nanoparticles, foaming agents, various polymers, emulsions, among others, as provided via the sampling device 110.

Referring now to FIGS. 3-8, various assay templates are provided, according to various aspects of the present disclosure. The assay template 300 may be comprised partially or entirely of a liquid-immobilizing material or media, such as, for example, an absorbent material or media 310, such that the extracted sample material from an egg may be absorbed into an absorbent portion of the assay template 300 when dispensed thereon. The liquid-immobilizing materials or media may include, for example, moisture-retaining materials (porous and alveolar in nature), gels (hydrogels, aerogels, xerogels), foam, cellulosic materials (such as various types of paper, woven fabrics or non-woven materials), microfluidic structures (honeycomb mesh), or other types of hydrophilic materials. The paper materials may include filter paper, office paper, chromatography paper, specialty papers, cardboard or any other suitable paper types capable of absorption. In some instances, Whatman paper of grades 1 and 3 may be used.

Such use of absorbent materials 310 may enable the bi-dimensional spread of an extracted sample material, thereby affording a large and easily-accessible surface area for further processing, enabling the use of small amounts of costly reagent. That is, the natural wicking of the absorbent cellulosic material may allow the sample material to spread horizontally, which enables the use of small reagent volumes (1-2 μl or less), faster heating of the assay location, especially when using radiation-based heating, higher-level functionalization of the cellulosic material for more complex assay formats (printing of channels, electrodes, etc.). Further, the bi-dimensionally spread sample may be interrogated in various sublocations on the absorbent material to take advantage of various properties of lateral-flow assay technology such as, for example, chromatographic separation of target molecules (DNA) from interfering matrix components, assay multiplexing for multiple single-nucleotide polymorphism (SNP) detection, genetic analysis, or other forms of bioanalysis (e.g., immunoassays, enzymatic activity detection), and signal enhancements due to specific interaction of signal molecules with the absorbent material. Moreover, the form of the absorbent material allows for easy handling and processing for conveyance, for assay processing, and for disposal (e.g., discard as solid waste for incineration).

Additionally, such absorbent materials may allow for convenient high-speed processing. In addition, the absorbent material, and particularly the cellulosic or paper materials may allow for a low-cost disposable material that can deliver cost efficiency with respect to the reagent. Further, the open format of cellulosic material may be forgiving of any vertical deviation of the sampling needle. In this regard, the sampling module 10 may be designed so that it can still operate with sampling needles that are bent and the paper-based assay template may be punctured by the sampling needle without damage to the needle/sampling tool or to the assay support or substrate. Moreover, cellulosic materials may be thin and easily fed through machines. In this regard, the thinness of cellulosic materials may enable small vertical clearances allowing for shorter sampling tools, thus enabling greater operation accuracy (X-Y positioning), affording better performance (sampling) and lower risk of damage (less downtime for repair). In addition, the thinness of cellulosic materials may make the assay template amenable to a variety of heating approaches, including infra-red lamps or resistive heating elements.

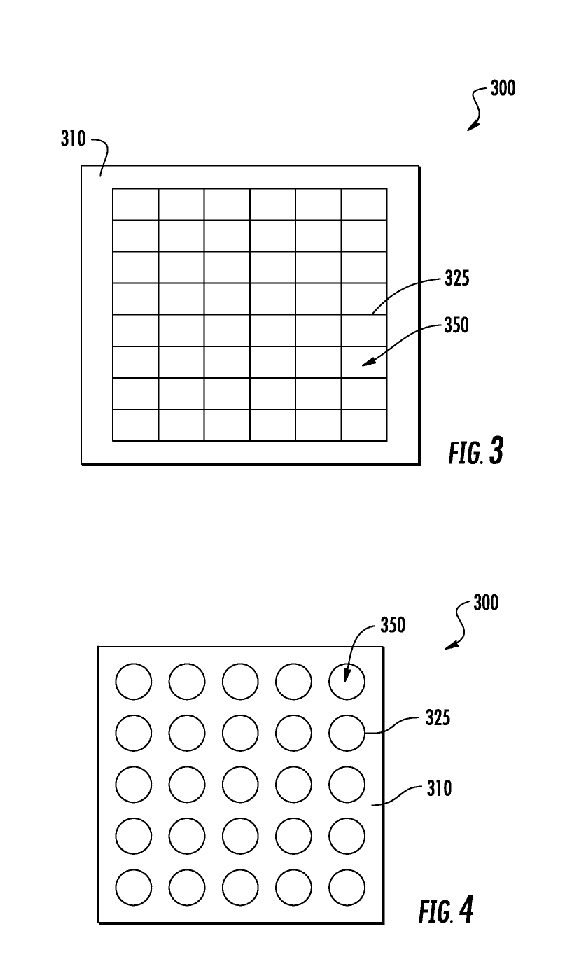

As shown in FIGS. 3 and 4, the absorbent material or media 310 may have a hydrophobic pattern 325 disposed thereon to create or define individual sample receptacles 350 or assay locations on the assay template 300. Such a hydrophobic pattern 325 may act as a barrier for containing a sample material dispensed onto the assay template 300 at a predefined location. The hydrophobic pattern may be applied to the assay template 300 in any suitable manner such as, for example, printing (e.g., inkjet printing), stamping, embossing, laser-structuring, stencil application, vacuum or atmospheric deposition, or other deposition forms, masking techniques, or other processes for treating a surface area such as coating. The hydrophobic pattern may be defined by hydrophobic materials or substances such as, for example, alkyl ketene dimer (AKD), alkenyl succinic anhydride (ASA), rosin, latex, silicones, fluorochemicals, ink, polyolefin emulsions, polymers, resin and fatty acids, natural and synthetic waxes, and any other hydrophobic substances known in the art.

In instances of applying a hydrophobic pattern, the assay template 300 may be comprised partially or entirely of a non-absorbent material or media, such as, for example, a polymer or plastic material or substrate (e.g., polyvinyl chloride (PVC) or polyethylene terephthalate (PET)). In this regard, the hydrophobic pattern may be applied to provide raised or bounding features to form a plurality of assay wells, such as by using hydrophobic ink to enable better discretization of assay locations on the non-absorbent surface. In addition, patterning with an additional hydrophilic ink/hydrogel on the footprint of each assay location may be provided so as to provide better droplet immobilization during lateral transport of an array or in-situ moisture retention. A blocking agent, such as, for example, bovine serum albumin (BSA), polyethylene glycol (and derivatives), hydrogels (alginate, acrylamides, agarose), may be provided on the non-absorbent surface to pre-block the surface such that it enables the assay process to run in the presence of a higher concentration of denaturant of the formamide family which, in turn, enables lower processing temperatures overall. In some instances, such non-absorbent substrate may be of a black or dark color so as to provide low optical background. In some instances, the non-absorbent substrate may be embossed with assay wells.

The hydrophobic pattern 325 may define the sample receptacles 350 in various arrays. Each sample receptacle 350 may be configured to receive a sample of material extracted from a respective egg, such as blood. Assay template 300 may have various configurations and arrays of hydrophobic patterns defining the sample receptacles 350 in accordance with aspects of the present disclosure. Material extracted from eggs may be disposed within respective sample receptacles 350 according to various dispensing patterns. In some instances, the dispensing pattern may be based upon the egg flat configuration. According to some aspects, the sampling devices 110 may be capable of condensing and/or expanding to align with the sample receptacles 350 for dispensing the extracted sample material thereon. Dispensing patterns may be controlled via a controller.

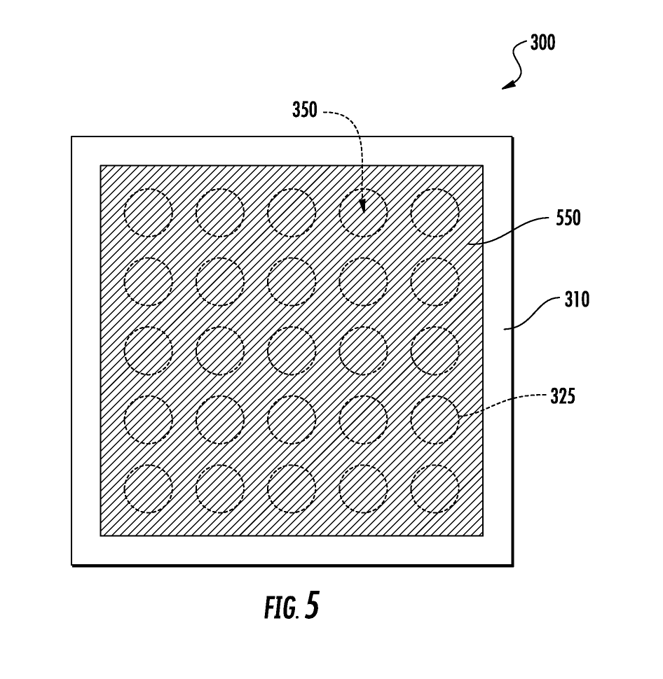

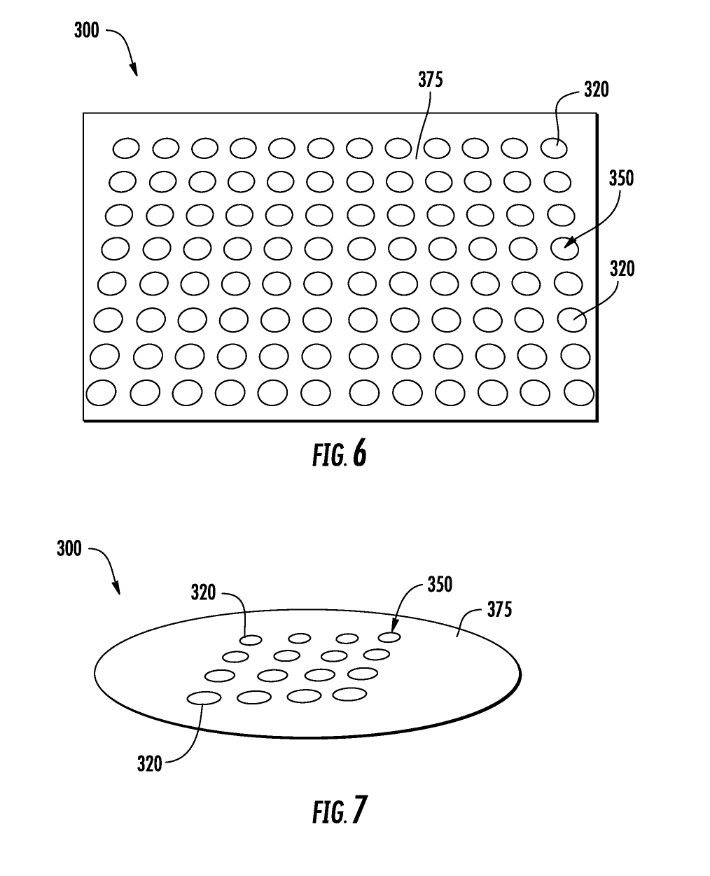

According to some aspects, the absorbent material 310 may form the substrate of the assay template 300, as shown in FIG. 1. That is, the absorbent material 310 may be a continuous sheet that extends throughout the sampling and assay modules for transporting the extracted sample material. In other instances, the absorbent material 310 may be discontinuous such that the absorbent material may be carried or a carrier substrate 600, as shown in FIG. 8. In this regard, a plurality of discrete assay templates 300 may be carried through the sampling and assay modules by the carrier substrate 600. In other instances, as shown in FIGS. 6 and 7, the absorbent material 310 may be provided as a plurality of hydrophilic pads 320 disposed on a hydrophobic substrate 375. In this manner, each hydrophilic pad 320 may act as an individual sample receptacle 350 or assay well or assay location. Although, each hydrophilic pad 320 may have the hydrophobic pattern 325 disposed thereon such that each hydrophilic pad 320 forms an assay template 300. It will be understood that the assay template 300 may have varying configurations with respect to the relationship between the absorbent material 310 and the hydrophobic pattern 325 and the means for moving the assay template 300 throughout the sampling and/or assay modules. For example, the assay template 300 may have assay arrays spread out to allow for a more cost-efficient array-support material between the discrete absorbent materials.

According to some aspects, the underside of a free-standing cellulosic material may be treated with an impermeable or hydrophobic coating in lieu of a plastic backing material. Such a coating may seal off the underside of the absorbent material to allow for better control of moisture at the assay location. Thin coatings may include spray-coated liquid materials, plasma-deposited hydrophobic coatings, printed hydrophobic inks, or the like.

Alternatively, the underside of the plastic backing material may be populated with the absorbent material or media 310 and accompanying hydrophobic pattern 325 disposed thereon such that the overall assay template 300 is two-sided. That is, the sample receptacles 350 may be populated on both sides of the assay template 300 such that both sides can received sample material, wherein the amount of assay template disposables created by the system is reduced by a factor of two.

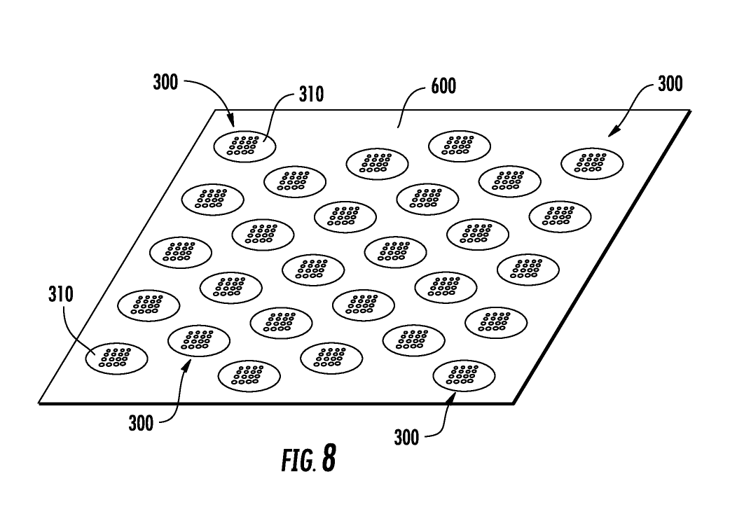

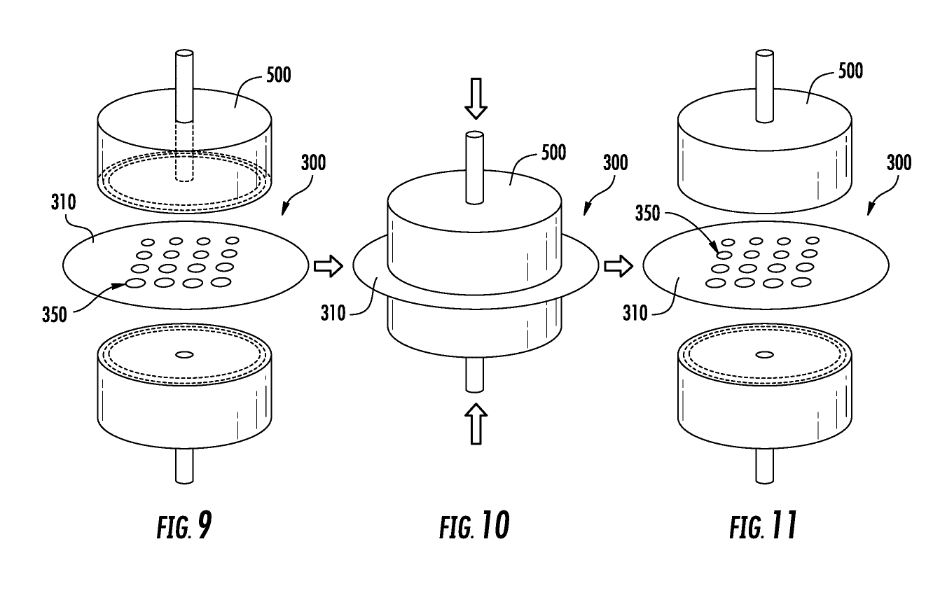

In some instances, the assay may need to be kept moist in order for accurate analysis in determining the characteristic. However, due at least partly to the small volumes of sample material extracted from the egg, it may be difficult to retain moisture in the assay. As such, this problem may be addressed in several ways. According to some aspects of the present disclosure, external moisture may be applied to the assay template via a humidity chamber or a moisture-introducing press 500, as shown in FIGS. 9-11. In some instances, an appropriate level of moisture present in the assay may be provided through the various steps of the assay process, including denaturation, incubation, and detection.

According to other aspects of the present disclosure, retention of internal moisture may be employed for keeping the assay moist. In some instances, as shown in FIG. 5, retention of internal moisture may be addressed by applying a film 550 (e.g., plastic) or a sealant over the assay location on the absorbent material of the assay template 300 after the reagent dispense in the assay protocol.

In other instances, retention of internal moisture may be addressed by the implementation of moisture retention modifiers such as high-boiling point liquids such as, ionic liquids, into the assay process. In this regard, the implementation of ionic liquids may be used to control evaporative loss on the absorbent cellulosic materials forming the assay template 300. In some instances, the reagent may be reformulated into a high-boiling point liquid capable of retaining its moisture during subsequent heating steps. According to some aspects, ionic liquids such as deep eutectic solvents (DES) may be used as the medium in which a molecular diagnostic assay is conducted on the cellulosic material forming the assay template 300 for the purpose of detecting the presence of specific DNA sequences at high-speed, low cost in the poultry industry. In some instances, moisture may be retained in the assay by adding a DES substance at a level of about 50% to about 80%. Such DES substances may include, for example, glycerol:choline, ethylene glycol:choline, malonic acid:choline, oxalic acid:choline or urea:choline, among many others.

Still in other instances, retention of moisture within the sample (e.g., whole blood) may be assisted by diluting the sample with a moisture retention modifier such as a solvent, such as, for example, a hydrophilic solvent. According to one particular aspect, dimethylformamide (DMF) may be used to dilute the sample material, which may be used to aid in low-temperature processing of the sample material. In such instances of diluting with DMF, for example, sample denaturation may be performed at about between 70-80° C., rather than the typical 95-120° C. Moreover, with the addition of DMF, incubation of the sample material may be performed at about 50° C., rather than the typical 65° C., which decreases moisture loss of the sample and may eliminate the need for external moisture supply or stringent humidity control.

Other moisture retention modifiers may include alkylformamides of high boiling points (boiling point>100° C.), such as, for example, N-Methyl formamide (MMF), formamide, and derivatives thereof.

Further, retention of moisture within the sample may be assisted by adding a thickening agent or substance to the sample. For example, sodium silicate or metasilicate may be added to the sample material so as to further improve the moisture retention properties of the overall assay template system.

An assay template handling system may be provided for moving the assay template 300 through the egg processing system. The assay template handling system may move the assay template 300 between the sampling module 10 and the assay module 20 such that extracted material from the eggs may be transported from the sampling module 10 to the assay module for determination of the characteristic. In this regard, the assay template handling system may be configured to move the sample receptacles 350 beneath respective sampling devices 110 such that material extracted from eggs can be dispensed within appropriate sample receptacles. The extracted material may then be moved and subjected to an assay protocol, as described further herein.

The assaying module 20 may be configured to process a the assay template 300 containing material extracted from eggs as described above in order to determine one or more characteristics of the eggs. In some instances, a holding area may be provided to receive and hold the assay template 300 containing material extracted from a plurality of eggs for a predetermined period of time. In some instances, the holding area may include a denature station 220 in which the extracted sample material is subjected to a denaturation process. The assay template may then be transferred from the holding area to a dispense station 200 where an assay reagent is added to the sample receptacles of the assay template. The assay template may then be transferred to an incubation station 230 in which the combined extracted sample material and reagent are subjected to incubation. After a predetermined period of time, the assay template may be transferred to the detector station 210 such that material in each sample receptacle may be analyzed to determine the characteristic.

A processing sequence according to aspects of the present disclosure will now be described with reference to FIG. 12. An absorbent assay template 300 is provided and moved beneath a sampling device 110 to receive a sample material (e.g., blood) extracted from eggs. The sample material may be dispensed substantially in the center of the sample receptacle. Within the assaying module 20, the absorbent assay template 300 may be moved via a conveyor system to the denature station 220 and then beneath a dispensing head 400 which dispenses a predetermined amount of reagent into each respective sample receptacle. The reagent may be dispensed off-center onto the sample receptacle. The absorbent assay template 300 may then progress through an environmentally-controlled chamber (e.g., incubation station 230) for a predetermined period of time. The absorbent assay template 300 may then be moved to the detector station 210 such material extracted from eggs may be analyzed to determine the characteristic. In some instances, a sample stacker may be provided to hold the samples between dispense of the sample material and the denature station 220. Such a sample stacker may have a first-in first-out configuration and protocol.

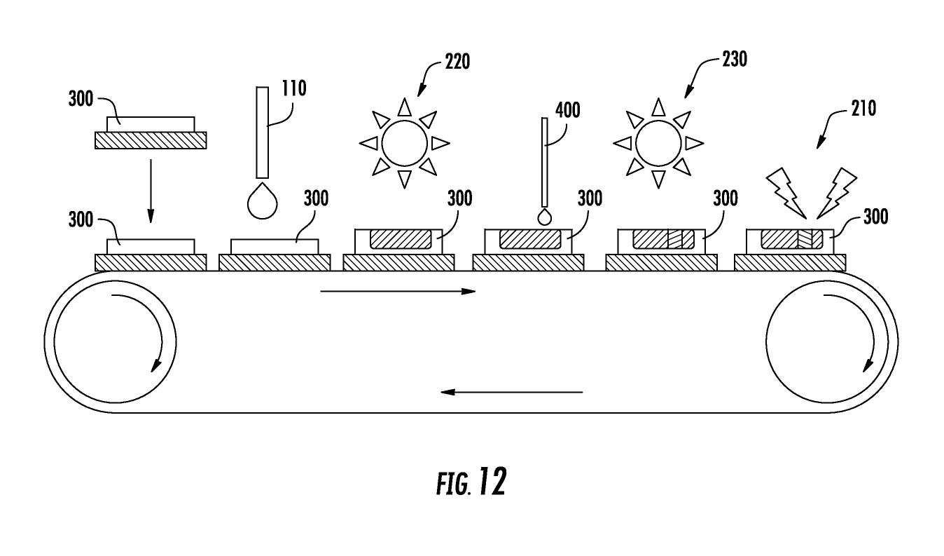

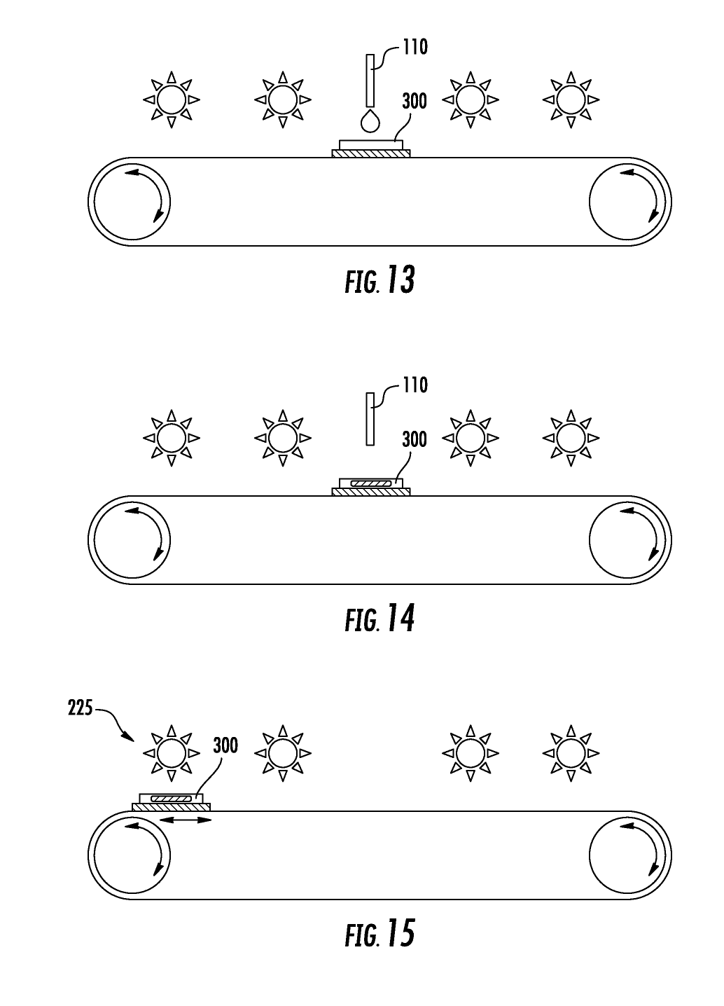

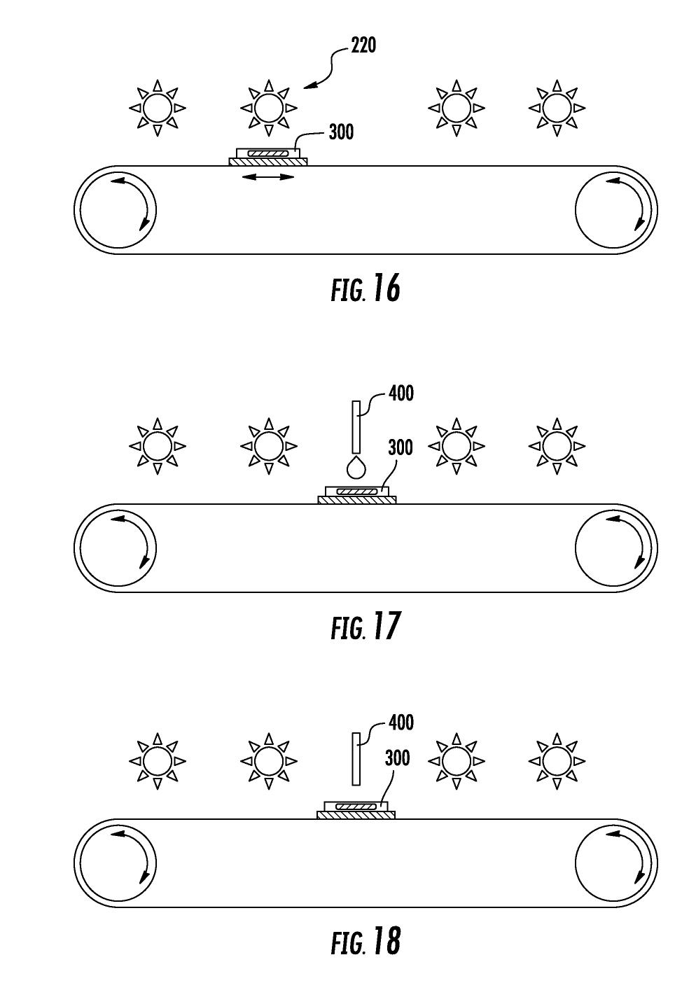

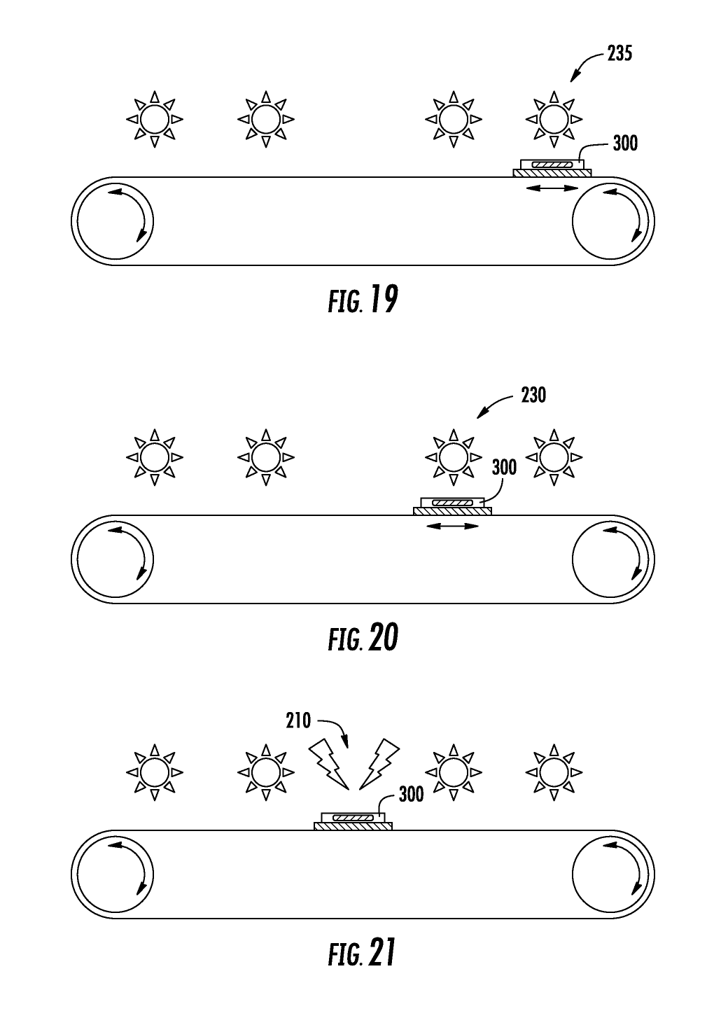

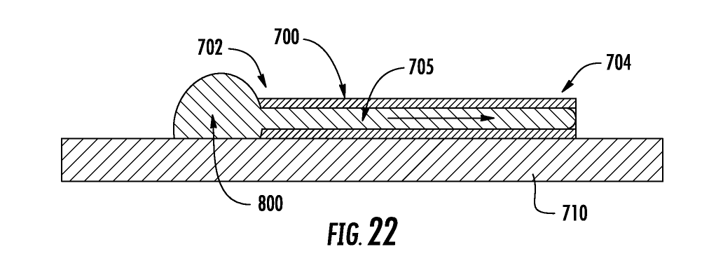

An alternative processing sequence according to aspects of the present disclosure will now be described with reference to FIGS. 13-21. As shown in FIG. 13, an absorbent assay template 300 is provided and moved beneath a sampling device 110 to receive a sample material (e.g., blood) extracted from eggs. As shown in FIG. 14, the sample material may be absorbed into the absorbent assay template 300. The absorbent assay template 300 may be moved via a linear actuator system to a ramp-up denature station 225 capable of ramping up the temperature in a short period of time, as shown in FIG. 15. The absorbent assay template 300 may then be moved to denature station 220, as shown in FIG. 16. As shown in FIG. 17, the absorbent assay template 300 may then be moved beneath a dispensing head 400 at the dispensing station 200 which dispenses a predetermined amount of reagent into each respective sample receptacle. The reagent may be absorbed into the absorbent assay template 300, as shown in FIG. 18. The absorbent assay template 300 may then move to a ramp-up incubation station 235 capable of ramping up the incubation temperature in a short period of time, as shown in FIG. 19. As shown in FIG. 20, the absorbent assay template 300 may then progress through an environmentally-controlled chamber (e.g., incubation station 230) for a predetermined period of time. The absorbent assay template 300 may then be moved to the detector station 210 such material extracted from eggs may be analyzed to determine the characteristic, as shown in FIG. 21.

The incubation and denature stations may have appropriate heating sources such as infrared lamps, according to aspects of the present disclosure. In some instances, atmospheric-plasma torches or systems or corona-discharge wands or systems (both operating near room temperature) may be used in place of infra-red lamps.

Aspects of the present disclosure are not limited to identifying gender of eggs. Various assaying techniques may be utilized for analyzing material extracted from eggs to identify various characteristics (e.g., gender, pathogen content, genetic markers related to bird health or performance) of eggs. For example, antibody-based systems and methods (e.g., commercial pregnancy testing systems and methods) may be utilized to detect estrogen in egg material. Moreover, antibody-based systems may be utilized to detect pathogens (e.g., salmonella and Marek's disease). As another example, PCR (polymer chain reaction) analysis may be utilized to detect the presence/absence of W chromosomes in egg material. Moreover, PCR analysis may be utilized to detect various genetic traits/flaws in egg material. Accordingly, assaying modules may be provided that facilitate pathogen detection and genetic analysis of avian eggs.

Referring now to FIG. 1, an assaying module 20, according to aspects of the present disclosure, that is configured to assay material extracted from eggs contained within sample receptacles in the assay template is illustrated. The illustrated module 20 includes a plurality of chambers, stations, or areas that are connected via conveyor systems that are configured to transport the assay template sequentially through the areas. In some instances, the chambers, stations, or areas may be maintained at predetermined temperature and humidity levels. Additional environmental controls may be utilized as well.

According to other aspects of the present disclosure, as illustrated in FIG. 22, an assay template may incorporate a capillary film implementing capillary action to transport sample material within the capillary film. In some instances, the capillary film may include a plurality of microfluidic conduits assembled in a planar space, such as, for example, in a ribbon form. Such a configuration may be used to maintain the sample material in a confined space such that it does not evaporate during various thermal steps such as denaturation and incubation. Referring to FIG. 22, a microfluidic conduit 700 (microtubes) may be coupled to a support substrate 710. The microfluidic conduit 700 may define a microfluidic path 705. The sample material 800 may be deposited proximate to a first end 702 of the microfluidic conduit 700 and then transported toward a second end 704 thereof via capillary action.

Many modifications and other aspects of the present disclosure set forth herein will come to mind to one skilled in the art to which this disclosure pertains having the benefit of the teachings presented in the foregoing descriptions and the associated drawings. Therefore, it is to be understood that the present disclosure is not to be limited to the specific aspects disclosed and that modifications and other aspects are intended to be included within the scope of the appended claims. Although specific terms are employed herein, they are used in a generic and descriptive sense only and not for purposes of limitation.

That which is claimed:

1. A method of processing avian eggs having an identified characteristic, comprising:
conveying a plurality of eggs to a sampling apparatus;
automatically extracting a sample material from the eggs using the sampling apparatus;
automatically depositing the extracted sample material onto a liquid-immobilizing assay template;
transporting the deposited sample material absorbed into the liquid-immobilizing assay template to an assaying station; and
automatically assaying the extracted sample material using the assaying station to identify eggs having a characteristic.

2. A method according to claim 1, wherein automatically depositing the extracted sample material onto a liquid-immobilizing assay template further comprises automatically depositing the extracted sample material onto an absorbent paper-based assay template formed of cellulosic material.

3. A method according to claim 2, wherein automatically depositing the extracted sample material onto an absorbent paper-based assay template further comprises automatically depositing the extracted sample material onto an absorbent paper-based assay template having a hydrophobic barrier pattern defining an array of assay locations.

4. A method according to claim 3, further comprising disposing the absorbent paper-based assay template on a carrier substrate configured to transport the extracted sample material for processing.

5. A method according to claim 2, wherein automatically depositing the extracted sample material onto an absorbent paper-based assay template further comprises automatically depositing the extracted sample material onto an absorbent paper-based assay template having a plurality of hydrophilic pads discretely arranged on a substrate to form an array of assay locations.

6. A method according to claim 1, further comprising dispensing an assay medium onto the liquid-immobilizing assay template, the assay medium being an ionic liquid configured to retain moisture at the absorbent assay template.

7. A method according to claim 6, wherein dispensing an ionic liquid onto the liquid-immobilizing assay template further comprises dispensing a deep eutectic solvent onto the liquid-immobilizing assay template.

8. A method according to claim 1, wherein automatically assaying the extracted material using the assaying station to identify eggs having a characteristic further comprises:
denaturing the extracted sample material;
dispensing an assay reagent onto the liquid-immobilizing assay template such that the extracted sample material and assay reagent form an assay sample;
sealing the assay sample; and
incubating the assay sample.

9. A method according to claim 1, wherein automatically extracting a sample material from the eggs comprises extracting material selected from the group consisting of amnion, shell, tissue, membrane and blood.

10. An egg processing system, comprising:
a sampling apparatus configured to extract sample material from a plurality of eggs;
a conveyor system configured to convey a plurality of eggs to the sampling apparatus;
a liquid-immobilizing assay template configured to receive and absorb the extracted sample material from the sampling apparatus;
an assay template handling system configured to position the liquid-immobilizing assay template proximate to the sampling apparatus for receiving the extracted sample material therefrom; and
an assaying station configured to assay the sample material extracted from the eggs so as to identify eggs having a characteristic.

11. The egg processing system of claim 10, wherein the liquid-immobilizing assay template comprises an absorbent paper-based assay template formed of cellulosic material.

12. The egg processing system of claim 11, wherein the absorbent paper-based assay template comprises a hydrophobic barrier pattern defining an array of assay locations.

13. The egg processing system of claim 12, wherein the assay template handling system comprises a carrier substrate, the absorbent paper-based assay template being disposed on the carrier substrate for transporting the extracted sample material during processing.

14. The egg processing system of claim 11, wherein the absorbent paper-based assay template comprises a plurality of hydrophilic pads discretely arranged on a substrate to form an array of assay locations.

15. The egg processing system of claim 10, further comprising a dispensing device configured to dispense an assay medium onto the liquid-immobilizing assay template, the assay medium being an ionic liquid configured to retain moisture at the liquid-immobilizing assay template.

16. The egg processing system of claim 15, wherein the ionic liquid comprises a deep eutectic solvent.

17. The egg processing system of claim 10, wherein the assay template handling system is further configured to transport the liquid-immobilizing assay template from the sampling apparatus to the assaying station.

18. The egg processing system of claim 10, further comprising a treatment station configured to treat the eggs based upon the identified characteristic.

19. The egg processing system of claim 10, further comprising a sorting station configured to sort the eggs based upon the identified characteristic.
